(19) United States
(12) Patent Application Publication
(10) Pub. No.: US 2024/0087690 A1
(43) Pub. Date: Mar. 14, 2024

(54) AN INDIVIDUALIZED GROUP TESTING METHOD FOR MEDICAL TEST SAMPLES

(71) Applicant: Synaptic, ApS, Odense C (DK)

(72) Inventor: Farzin KAMARI, Odense C (DK)

(21) Appl. No.: 17/924,903

(22) PCT Filed: May 12, 2021
(86) PCT No.: PCT/DK2021/050150

§ 371 Date: Nov. 11, 2022

(30) Foreign Application Priority Data
May 12, 2020 (NO) 20200557

(51) Int. Cl. G16H 10/40; G16B 25/10; G16B 40/20

(52) U.S. Cl. CPC G16H 10/40; G16B 25/10; G16B 40/20

(57) ABSTRACT

The invention relates to a method for group testing of medical test samples for detecting a target pathology indicator, wherein each of said medical test samples stems from an associated undiagnosed individual, said method comprising the steps of: pre-estimating a potential pathology indicator load of said target pathology indicator in each of said medical test samples at least partly based on individualized data of said associated undiagnosed individual, wherein said potential pathology indicator load is indicative of an actual pathology indicator load of said target pathology indicator when said target pathology indicator is present; pooling a subset of said medical test samples into a test pool at least partly based on said potential pathology indicator load of each of said medical test samples to reduce a potential pathology indicator load variance of said test pool; and detecting said target pathology indicator by group testing said test pool.

### FIELD OF THE INVENTION

The present invention relates to a group testing method for medical test samples. The invention further relates to a group testing system. Furthermore, the invention relates to a pooling plate for facilitating group testing.

### BACKGROUND OF THE INVENTION

Medical group testing permits a large number of medical test samples to be rapidly evaluated to detect the presence of a target pathology indicator, such as a virus, in the test samples. In group testing, several test samples are mixed together in a test pool and are tested as if it was a single test. If the presence of the target pathology indicator is not detected in the test pool, i.e. the test is negative, all the individual medical test samples in test pool can be assessed as not containing the target pathology indicator. If instead the target pathology indicator is detected in the test pool, i.e. the test is positive, the individual medical test samples may be tested on an individual basis to identify in which of the samples the target pathology indicator is present.

Group testing may thus permit far fewer tests, than if all medical test samples were to be tested individually. This is especially the case if the fraction of medical test samples in which the target pathology indicator is expected to be present is low, since then, testing medical test samples individually is rarely necessary.

However, group testing also has drawbacks. When several medical test samples are mixed together, in which one or a few of the medical test samples contains the target pathology, the concentration/load of the target pathology indicator in the resulting admixture is diluted. Due to this dilution, the risk of not detecting a present target pathology indicator is increased for every medical test sample added into the test pool.

Generally, it is desirable to further reduce the number of test necessary in context of testing and group texting. Particularly without having a large risk of not detecting a target pathology indicator.

### SUMMARY OF THE INVENTION

The inventors have identified the above-mentioned problems and challenges related to group testing, and subsequently made the below-described invention which may improve group testing of medical test samples.

The invention relates to a method for group testing of medical test samples for detecting a target pathology indicator, wherein each of said medical test samples stems from an associated undiagnosed individual, said method comprising the steps of:
pre-estimating a potential pathology indicator load of said target pathology indicator in each of said medical test samples at least partly based on individualized data of said associated undiagnosed individual, wherein said potential pathology indicator load is indicative of an actual pathology indicator load of said target pathology indicator when said target pathology indicator is present;
pooling a subset of said medical test samples into a test pool at least partly based on said potential pathology indicator load of each of said medical test samples to reduce a potential pathology indicator load variance of said test pool; and
detecting said target pathology indicator by group testing said test pool.

In typical group testing, prior to performing the step of detecting the target pathology indicator by group testing the test pool, it is typically unknown whether the target pathology indicator is present in the medical test samples or not. Further, if the target pathology indicator is indeed present, the actual pathology indicator load is unknown. When mixing several test samples, an actual pathology indicator load may be diluted. If too many medical test samples are included in the test pool, the resulting pathology indicator load may be reduced below a detection threshold, i.e. a limit of detection, where certainty of detecting the target pathology indicator is compromised. As a consequence, it is difficult to include many medical test samples in the test pool.

By utilizing individualized data of undiagnosed individuals associated with medical test samples, a pre-estimation of the potential pathology indicator load is possible. In turn, by having a pre-estimation of the potential pathology indicator load, which is indicative of the actual pathology indicator load when the target pathology indicator is present, it is possible to optimize the sample number of medical test samples in a test pool, which is advantageous.

In an exemplary embodiment of the invention, medical test samples are group tested the target virus SARS-CoV-2. A symptomatic individual infected with SARS-CoV-2 may have a larger actual virus load than an infected individual without symptoms. Hence, the potential virus load of an individual with symptoms of COVID-19 may typically be larger than the potential virus load of an individual without symptoms of COVID-19. In the embodiment, medical test samples are then pooled based on whether each associated individual has symptoms of COVID-19 or not. Since the actual virus load of a positive medical test sample of a person with symptoms of COVID-19 may be larger than the actual virus load of a positive medical test sample of a person without symptoms of COVID-19, the test pools of medical test samples from persons with symptoms of COVID-19 may have a larger sample number than test pools with medical test samples from persons without symptoms, while advantageously minimizing the risk a false negative group test due to dilution. In simple terms, the invention may permit a larger number of samples in group testing which is advantageous. As a result, the number of tests needed for a certain group of people may be reduced.

In an alternative embodiment, a test pool based on medical test samples from individuals with symptoms is smaller than a test pool based on medical test samples from individuals without symptoms.

Generally, an object of the invention is to improve group testing of medical test samples. A method of the invention may also be understood as an assay.

More specifically, the invention may permit utilizing individualized data of individuals associated with the group test to tailor and adjust the group testing scheme to various conditions including the potential pathology indicator load. The invention may thus introduce personalized or individualized group testing.

The target pathology indicator may be understood as a medical indicator of a disease of an individual, which may be identifiable in a medical test sample obtained from that individual. Examples include pathogens, such as a virus, bacterium, protozoan, prion, viroid, or fungus. The pathology indicator is not restricted to being, e.g. the pathogen itself, but may for example be an analyte which indicate the presence of a pathogen or a disease in the individual.

The actual pathology indicator load may be understood as the load of the target pathology indicator in the medical test sample in case the sample is positive. For example, if the target pathology indicator is pathogenic bacteria, such as Mycobacterium tuberculosis bacteria, the actual pathology indicator load may be the quantity of these bacteria (or an indicator) actually present in a positive medical test sample. Or, if the target pathology indicator is a pathogenic virus, the actual pathology indicator load may be the viral load in a positive medical test sample. In the context of this invention, the “load” may also be understood as a “concentration”, “abundance”, or a “quantity”.

The potential pathology indicator load may be understood as an estimate of the actual pathology indicator load, assuming that a given medical test sample is positive, i.e. assuming that the target pathology indicator is present in the medical test sample. Hence, the potential pathology indicator load may not depend on whether or not the target pathology indicator load is present or not.

The step of pooling a subset of the medical test samples may be understood as determining or selecting a subset of the medical test samples which have to be mixed such that these medical test samples can be group tested as if they were a single test. E.g., given 100 medical test samples, 20 of these may be pooled, such that these 20 medical test samples can be tested collectively.

Individualized data may be understood as data associated with the undiagnosed individual, which may at least be indicative of a potential pathology indicator load or an actual pathology indicator load. The data may typically be non-personal, in the sense that the individual may remain anonymous as the medical test sample of that individual is tested. However, note that some embodiments of the invention may use non-anonymous data as well. Individualized data may also be referred to as personalized data, or personal data.

A medical test sample stems from an undiagnosed individual. That undiagnosed individual is thus associated with the medical test sample. A medical test sample may also be referred to as a biological test sample or a medical/biological specimen. Embodiments of the invention may typically involve several medical test samples, wherein each respective medical test sample of the medical test samples stems from a respective associated undiagnosed individual from a group of undiagnosed individuals. Non-limiting examples of medical test samples include samples of body fluids, tissue samples, secretion, excretion, etc. Examples of secretion include serous fluids, mucus, saliva, etc. Examples of excretion include urine and faeces. Examples of body fluids include blood samples, cerebrospinal fluid, peritoneal fluid, pleural fluid, amniotic fluid, etc. Blood samples may refer to samples of whole blood or blood components such as plasma/serum. As for blood, the medical samples may also be an extracted component of one or more samples extracted from the body. It is noted that some of the above examples may somewhat overlap and may in cases therefore not be considered alternatives.

In some embodiments, undiagnosed individuals are humans. In some embodiments, undiagnosed individuals are other living species, such as animals.

According to embodiments of the invention, the medical test samples are pooled to reduce the variance of the potential pathology indicator load. That is, the variance of the potential pathology indicator load is reduced for the pooled test pool in comparison with the variance of the potential pathology indicator load of all of the medical test samples. The variance may be understood as a statistical variance, i.e. the expectation of the squared deviation of a variable from its mean. In the context of the invention, the variable may then be the potential pathology indicator load.

The variance VAR for n values of the variable xi may be parameterized as

$$VAR = \frac{1}{n} {\sum }_{i = 1}^{n} {( x i - u )}^{2} ,$$

where the average value u may be expressed as

$$u = \frac{1}{n} {\sum }_{i = 1}^{n} x i .$$

Note that embodiments of the invention do not have to rely on explicitly calculating the variance of the potential pathology indicator load, but merely performing pooling in manner which reduces that variance.

The step of detecting the target pathology indicator may be understood as performing a detection procedure collectively on the test pool, e.g. conventional group testing of the test pool. Exemplary outcomes of the step of detecting the target pathology indicator are “positive” and “negative”. In other words, the target pathology indicator was identified in the test, or it was not identified. Thus, the step of detecting the target pathology indicator may not result in actually finding it, particularly if the target pathology indicator is not present.

In an embodiment, the method of the invention includes a step of providing or receiving the medical test samples. Accordingly, this does not include the extraction of the sample(s) from the associated undiagnosed individual. Thus, the medical test samples may be considered pre-extracted, and the extractions are not part of the method of this embodiment.

In another embodiment, the method includes a step of establishing or extracting each medical test sample of the medical test samples from the associated undiagnosed individual.

In embodiments of the invention, said test pool has a sample number of said medical test samples in said test pool.

The test pool may for example have a sample number of 3, 5, 10, 20, or more than 20 medical test samples.

In embodiments of the invention, said sample number is at least partly based on said potential pathology indicator load.

By selecting the sample number based on the potential pathology indicator load, the sample number can be maximized while minimizing the risk for having a combination of actual pathology indicator load and sample number which falling under a limit of detection of the target pathology indicator, which is advantageous.

In embodiments of the invention, said sample number is at least partly based on a predefined amount of available test resources.

Test resources may be understood as apparatus, chemicals, machinery, and/or labour resources for performing the actual tests of the test pools. Examples of test resources are test kits, work hours, thermal cyclers, PCR plates, antibodies for binding to an antigen for the test, etc.

The test resources may simple put a constraint as to how many tests can be performed within some durations, e.g. 100 PCR tests for a target pathology indicator per day. If 1000 medical test samples have to be tested using 100 PCR tests, the average sample number of test pools may for example be selected to be approximately 10 medical test samples. Here, some of the test pools may typically have a sample number larger than 10 medical test samples, whereas some other test pools have a sample number smaller than 10 medical test samples.

The method may further take into account reserving test resources to a second test round for cases of a positive test, or take into account reserving test resources for quality control. For the exemplary numbers of 100 PCR tests for 1000 medical test samples, this may for example result in an average sample number of 20 or more than 20 medical test samples.

Basing the sample number on a predefined amount of available test resources may thus permit an improved utilization of test resources, which is advantageous.

In embodiments of the invention, each of said medical test samples in said test pool contribute with a sample volume to said test pool.

Each medical test sample may for example be (dissolved in) 1 millilitre of fluid. And, for example, of this 1 millilitre, 100 microlitres may be separated out for mixing with parts of other medical test samples.

In embodiments of the invention, said sample volume is at least partly based on said sample number.

The sample volume may be based on the sample number, e.g. a sample number of 10 may lead to a sample volume of 100 microlitres, and a sample number of 5 may lead to a sample volume of 200 microlitres, such that the volume of the final test pool is always 1 millilitre. Thus, by basing the sample volume on the sample number, the volume of the final test pool may be controlled, which is advantageous.

In embodiments of the invention, said sample volume is at least partly based on said potential pathology indicator load.

The sample volume may typically be proportional to the quantity of the amount/quantity of the actual pathology indicator which is brought to the test pool, and may thus influence whether detection of the target pathology indicator is possible. Consequently, basing the potential pathology indicator load on the sample volume is advantageous.

In embodiments of the invention, said step of pooling a subset of said medical test samples is at least partly based on retaining said potential pathology indicator load of each of said medical test samples of said test pool at least at a potential load dilution threshold.

As several medical test samples are mixed, each individual sample may be diluted, potentially increasing the risk of not detecting the target pathology indictor even if is present, i.e. a false negative.

By having the potential pathology indicator load being above a potential load dilution threshold, the risk of a false negative is minimized, which is advantageous.

The potential load dilution threshold may for example be some fraction of a potential pathology indicator load of a medical test sample. If, for example, the potential load dilution threshold is 10 percent of the potential pathology indicator load, the medical test sample should not be diluted by at most a factor of 10.

In embodiments of the invention, said potential pathology indicator load of a first medical test sample of said medical test samples is further based on said individualized data of said associated undiagnosed individual associated with a second medical test sample of said medical test samples, wherein said first medical test sample and said second medical test sample are different medical test samples.

The pre-estimation of a potential pathology indicator can for a certain medical test sample can thus potentially be based on individualized data associated with individuals of other medical test samples.

Individualized data of a group of individuals may thus affect the potential pathology indicator load of a single individual.

Having a potential pathology indicator load of a medical test sample being at least partly based on individualized data of an individual associated with another medical test sample is advantageous, since some pathologies may influence groups of people.

In embodiments of the invention, said medical test samples are at least 20 medical test samples, such as at least 50 medical test samples, such as at least 95 medical test samples, for example at least 200 medical test samples.

Having a certain minimal number of medical test samples ensures that the individual medical test samples can be pooled optimally, which is advantageous.

In embodiments of the invention, said medical test samples are based on swab samples, for example based on anterior nares swabs, mid-turbinate swabs, nasopharyngeal swabs, or oropharyngeal swabs.

A swab may be understood as a wad of absorbent material for removing material from an area. The material may for example be curled/wound around one end of a small stick.

In embodiments of the invention, said medical test samples are based on saliva samples.

In embodiments of the invention, said medical test samples are based on blood samples.

For example, plasma or serum of blood may be based on blood samples.

In embodiments of the invention, said medical test samples are based on tissue samples.

In embodiments of the invention, said medical test samples are based on urine or faeces samples.

In embodiments of the invention, said step of detecting said target pathology indicator by group testing said test pool is based on testing an admixture of said medical test samples in said test pool.

In embodiments of the invention, said step of detecting said target pathology indicator is based on a nucleic acid test, for example a nucleic acid amplification test.

A nucleic acid test may be understood as a technique for detecting a particular nucleic acid sequence, e.g. to detect a particular virus or a bacteria. Nucleic acid amplification tests may typically rely on amplification of genetic material by copying it. Examples of nucleic acid amplification tests are polymerase chain reaction (PCR), strand displacement assay, and transcription mediated assay (TMA).

In embodiments of the invention, said step of detecting said target pathology indicator is a step of repetitively performing polymerase chain reaction cycles on said test pool to detect said target pathology indicator.

A polymerase chain reaction test relies on thermal cycling, in which reactants (e.g. the admixture of the test pool) are exposed to repeated cycles of heating and cooling to permit DNA melting and enzyme-driven DNA replication, respectively. Thus, for each polymerase chain reaction cycle, the quantity of nucleic acids is doubled, which enable detection of initially faint amounts of material.

Polymerase chain reaction according to the invention may also be variants of PCR, such as reverse transcription polymerase chain reaction, two-tailed PCR, ligation-mediated PCR, methylation-specific PCR etc.

In embodiments of the invention, said step of repetitively performing polymerase chain reaction cycles is performed using a thermal cycler.

A thermal cycler may also be understood as a thermocycler, a PCR machine, or a DNA amplifier (although some machines may also be able to amplify RNA).

In embodiments of the invention, said step of detecting said target pathology indicator comprises performing blotting, such as western blotting, southern blotting, northern blotting, southwestern blotting, or dot blotting.

Western blotting is a widely used analytical technique to detect specific proteins in a sample.

In embodiments of the invention, said step of detecting said target pathology indicator comprises performing enzyme-linked immunosorbent assaying.

Enzyme-linked immunosorbent assay (ELISA) is a commonly used analytical biochemistry assay for detecting the presence of a ligand, e.g. a protein.

In embodiments of the invention, said polymerase chain reaction cycles have a predefined cycle threshold at least partly based on said potential pathology indicator load of medical test samples in said test pool.

When testing a sample using real time polymerase chain reactions, a cycle threshold may be introduced. The cycle threshold sets a limit to the number of repeated thermal cycles of heating and cooled before a deciding that a test is negative. If the target pathology indicator is found before the cycle threshold is crossed, the test is positive, otherwise the test is negative. Having such a cycle threshold may for example minimize the risk of amplifying contaminants, leading to false positive tests.

Cycle thresholds may for example be 10 thermal cycles, 20 thermal cycles, 30 thermal cycles, or 40 thermal cycles.

The potential pathology indicator load indicates the number of thermal cycles necessary to identify the target pathology indicator. If the actual pathology indicator load is large, fewer thermal cycles are required, than if the actual pathology indicator load is small. In a simple embodiment, doubling the potential pathology indicator load reduces the cycle threshold by one thermal cycle.

In embodiments of the invention, said cycle threshold is at least partly based on said sample number.

As more medical test samples are pooled, a medical test sample in which the target pathology indicator is present is diluted in the admixture of the test pool of the medical test samples. Thus, when increasing the sample number, it may be advantageous to also increase the cycle threshold, to ensure detection. In a simple embodiment, doubling the sample number increases the cycle threshold by one thermal cycle.

In some embodiments, the cycle threshold is determined based on both the potential pathology indicator and the sample number. Taking both of these factors into account may improve utilization of test resources while minimizing risk of false positive and/or false negative tests, which is advantageous.

In embodiments of the invention, said step of repetitively performing polymerase chain reaction cycles is based on real-time monitoring of polymerase chain reactions.

Real-time polymerase chain reactions may also be understood as quantitative polymerase chain reactions. Here, the amplification of the target pathology indicator is monitored during the test. Thus, the target pathology indicator may be observed earlier, which is advantageous.

In embodiments of the invention, said target pathology indicator is a target pathogen, said potential pathology indicator load is a potential pathogen load, and said actual pathology indicator load is an actual pathogen load.

In embodiments of the invention, said target pathology indicator is a target virus, said potential pathology indicator load is a potential viral load, and said actual pathology indicator load is an actual viral load.

In embodiments of the invention, said target pathology indicator is a target cancer indicator, said potential pathology indicator load is a potential cancer indicator load, and said actual pathology indicator load is an actual mutation indicator load.

In embodiments of the invention, said target pathology indicator is selected from virus, bacterium, protozoan, prion, viroid, fungus, or any combination thereof.

The invention may thus be utilized for group testing many various pathology indicators. Various examples of applications and target pathology indicators are Alzherimer's disease; animal diseases such as bovine viral diarrhea virus, mycobacteria, Bonamia ostreae, avian influenza; screening of blood donations for hepatitis C virus, hepatitis B virus, and HIV; Chlamydia; COVID-19; drug discovery; genetic screening; genotyping assays; gonorrhea; hepatitis B virus; hepatitis C virus; HIV; human papillomavirus; Listeria monocytogenes; malaria; nasopharyngeal bacteria; prevalence estimates; screening for rare diseases/mutations; sequencing and genome projects; and tuberculosis.

In embodiments of the invention, said step of pooling a subset of said medical test samples comprises that said potential pathology indicator load variance is reduced in said test pool in comparison with said potential pathology indicator load variance in said medical test samples.

By reducing the variance VAR of the potential pathology indicator load in the test pool in comparison with the variance VAR of the potential pathology indicator load in the entire ensemble of medical test samples, it is possible to optimize group testing, which is advantageous.

In embodiments of the invention, said step of pooling a subset of said medical test samples comprises that said potential pathology indicator load variance is reduced by at least 10 percent, for example at least 20 percent, for example at least 30 percent, for example at least 40 percent, such as at least 50 percent.

Such a reduction may be calculated relatively to the variance in all medical test samples.

In embodiments of the invention, said step of pooling a subset of said medical test samples comprises that an entropy of said potential pathology indicator load is reduced in said test pool.

In embodiments of the invention, said step of pooling a subset of said medical test samples comprises that an entropy of said potential pathology indicator load is reduced in said test pool in comparison with an entropy of said potential pathology indicator load in said medical test samples.

In context of the present invention entropy interpreted from a viewpoint of information theory. Thus, the entropy may also be understood as Shannon entropy.

The entropy ENT for n possible outcomes xi may be quantified as

$$ENT = - {\sum }_{i = 1}^{n} P ( xi ) log P ( xi ) ,$$

where P(xi) is the probability of the outcome xi and log is the logarithm.

Here, for example, the outcome may relate to the potential pathology indicator load, and the probability may relate to the probability of having a medical test sample with a certain potential pathology indicator load, e.g. within a certain range of potential pathology indicator loads.

Alternatively, the probability P may relate to a positive pathology indicator probability for a medical test sample, and consequently, the outcome may relate to a positive pool pathology indicator probability for a test pool.

The entropy may be calculated for a single test pool, or for an ensemble of test pools, each based on different medical test samples.

Generally, the entropy is lower if the outcome is easy to predict, and the entropy is higher if the outcome is harder to predict.

In embodiments of the invention, a subset of the medical test samples is pooled to reduce the entropy of the potential pathology indicator load of the test pool, e.g. relatively to the potential pathology indicator load of all of the medical test samples. This may ensure an improved pooling scheme, which is advantageous.

In some examples, a subset of the medical test samples is pooled into a test pool at least partly based on the potential pathology indicator load of each the medical test samples to reduce the entropy of the potential pathology indicator in the test pool without necessarily reducing the variance.

In embodiments of the invention, said test pool is a first test pool, wherein said method further comprises a step of pooling a subset of said medical test samples into a second test pool at least partly based on said potential pathology indicator load of each of said medical test samples, such that a first average potential pathology indicator load of medical test samples of said first test pool and a second average potential pathology indicator load of medical test samples of said second test pool are different.

By having several different test pools, group testing can be improved further, which is advantageous. Namely, each of the different test pools can be established based on varying potential pathology indicator loads of the medical test samples.

In embodiments of the invention, said first average potential pathology indicator load is larger than said second average potential pathology indicator load.

By having different average potential pathology indicator loads, the test pools may be individualized for optimization, which is advantageous.

In embodiments of the invention, said step of detecting said target pathology indicator comprises group testing said first test pool and said second test pool, respectively.

Typically, each established test pool will be tested, e.g. based on PCR, ELISA, western blot, or any other detection scheme/assay, e.g. as described within this disclosure. In an exemplary embodiment, the target pathology indicator is detected in the first test pool by group testing the first test pool and the target pathology indicator is detected in the second test pool by group testing the second test pool.

In embodiments of the invention, said sample number is a first sample number, wherein said second test pool has a second sample number of medical test samples in said second test pool, wherein said first sample number is larger than said second sample number.

By having different sample numbers, the test pools may be individualized for optimization.

In embodiments of the invention, said first test pool as associated with a first predefined cycle threshold of said polymerase chain reaction cycles, wherein said second test pool is associated with a second predefined cycle threshold of said polymerase chain reaction cycles, wherein said second cycle threshold is larger than said first cycle threshold.

By having different cycle thresholds, the test pools may be individualized for optimization.

By having the second cycle threshold being larger than the first cycle threshold, the cycle thresholds may for example match the first average potential pathology indicator load being larger than the second average potential pathology indicator load, which is advantageous.

In embodiments of the invention, said individualized data comprises demographic data.

Examples of demographic data are nationality, ethnicity, sociology, economy, and geography such as postal code.

Some disease may be more frequent, more severe, or lead to higher actual pathology indicator loads in certain demographic groups, and accounting for demographic data is thus advantageous.

In embodiments of the invention, said individualized data comprises age.

In embodiments of the invention, said individualized data comprises sex.

In embodiments of the invention, said individualized data comprises individualized health data.

The individualized health data may further comprise medical history, medication history, habit history such as smoking habits, etc.

In embodiments of the invention, said individualized health data comprises disease symptoms.

In embodiments of the invention, said individualized health data comprises condition severity.

In embodiments of the invention, said individualized health data comprises laboratory tests.

Laboratory tests may for example include an erythrocyte sedimentation rate tests, C-reactive protein tests, lymph count tests, etc. Such test may by indicative of a potential pathology indicator load and/or a positive pathology indicator probability and are thus advantageous to include in estimations.

In embodiments of the invention, said individualized data comprises medical test sample procedure details.

Test sample procedure details may for example to relate at what time a given medical test sample was obtained, whether obtained by a professional or by self-testing by the individual, the type of test used to obtain the medical test sample, etc.

Aspects like self-testing and time since the medical test sample was obtained may increase error or uncertainties when detecting the target pathology indicator by group testing the test pool. Thus, taking such factors into account, e.g. by reducing a sample number, is advantageous for reducing test uncertainties.

In embodiments of the invention, said step of pooling a subset of said medical test samples is at least partly based on a geographical pathology rate or a demographical pathology rate.

A geographical or demographical pathology rate may be understood as an indication of a rate of occurrence of a disease within a certain population group.

The relevance of the above-presented aspects relating to individualized data may be exemplified in context of the COVID-19 pandemic. Here, outbreaks have often occurred more strongly within certain demographic groups, the actual pathology indicator load (the actual virus load) is generally larger for symptomatic individuals, an infection is more severe in regard to health for some age groups, etc.

Even though the relevance of aspects of the invention may be exemplified in context of COVID-19, the invention may be utilized in many other contexts and for many other diseases or pathologies.

Generally, utilizing one or more particular sets of individualized data as exemplified above may improve predictability of the potential pathology indicator load, and is thus advantageous.

In embodiments of the invention, said individualized data comprises animal data.

In embodiments of the invention, said animal data is selected from behaviour, colour, voice, movement, species, or any combination thereof.

Embodiments of the invention may also be applicable to group test animal populations for a target pathology indicator. The animals may for example be fish such as salmon, Bovidae such as sheep and cattle, pigs, birds such as poultry, etc. In such cases, the individualized data may refer animal behaviour, colour etc. Animal behaviour may change with some pathologies.

The various examples of individualized data presented in this disclosure may be relevant depending on the chosen target pathology indicator. For one target pathology indicator, one type of individualized data may be suitable for accurately and/or precisely pre-estimating a potential pathology indicator, whereas for another target pathology indicator, another type of individualized data may be suitable.

In embodiments of the invention, said potential pathology indicator load is indicative of said actual pathology indicator load of said target pathology indicator when said target pathology indicator is present at least partly based on an estimated correlation between said individualized data and said potential pathology indicator load.

An estimated correlation may for example be based on a qualified guess or an estimate. It does thus not have to rely on actual data of a pathology. For example, experience from other pathologies may be used to estimate a correlation.

Introducing an estimated correlation between the individualized data and the potential pathology indicator load may provide improved group testing, which is advantageous.

In embodiments of the invention, said potential pathology indicator load is indicative of said actual pathology indicator load of said target pathology indicator when said target pathology indicator is present at least partly based on a pre-established correlation between said individualized data and said potential pathology indicator load.

If data relating to a target pathology and/or a related target pathology indicator, this data may be used for a pre-established correlation between individualized data and the potential pathology indicator load. Consequently, group testing according to the invention may be improved, which is advantageous.

In embodiments of the invention, said pre-established correlation is at least partly based on correlating historical individualized data with historical actual pathology indicator loads.

Historical individualized data may be understood as individualized data obtained in obtained previously. It does thus not necessarily relate to the individualized data of the individuals associated with the medical test samples. Instead, it may stem from scientific studies, mass testing of populations, previous implementations of the method of the invention, or other testing schemes capable of providing relevant data.

In embodiments of the invention, said historical individualized data comprises at least some of the same types of data as said individualized data.

These same types of data may for example be any of the data presented in this disclosure, e.g. demographic data, health data, age, etc.

In embodiments of the invention, said step of pre-estimating said potential pathology indicator load is performed using a machine learning algorithm trained using said historical individualized health data with historical actual pathology indicator loads.

In embodiments of the invention, said step of pooling a subset of said medical test samples is performed using a machine learning algorithm trained to at least partly reduce said potential pathology indicator load variance of said test pool.

In embodiments of the invention, said pre-established correlation is established based on a machine learning algorithm.

Machine learning has may be a powerful tool for analysing, implementing, and utilizing complex correlations, and is thus advantageous to use for pre-estimating the potential pathology indicator load and establishing the pre-established correlation between individualized data and the potential and/or actual pathology indicator load. It is further advantageous to use machine learning for pooling the medical test samples in one or more test pools, i.e. determining how the individual medical test samples should be distributed, for example to reduce variance or entropy in the test pools.

Various embodiments of the invention may have any combination of the above-suggested uses of machine learning algorithms. They may be implemented as a single algorithm or separate algorithms (e.g. a first, a second, and a third machine learning algorithm) which performs pre-estimation, pooling, and establishment of the pre-established correlation. Such separate algorithms can both be entirely decoupled and function independently of each other, or be coupled and feed information, such as data or trained correlations, to each other.

For implementation of machine learning, supervised learning may typically be used, although unsupervised learning or reinforcement learning may also be applicable. As such, the invention is not restricted to a particular type of machine learning.

For embodiments where the pre-estimation of a potential pathology indicator load is performed based on a machine learning algorithm, the machine learning algorithm may for example have trained a decision tree algorithm, or a support-vector network to perform the prediction, based on the pre-established correlation. Other possible examples of algorithms are cluster analysis, automated machine learning, linear regression, logistic regression, naive Bayes, linear discriminant analysis, k-nearest neighbour algorithm, neural networks (Multilayer perceptron), or similarity learning.

Note further that embodiments of the invention are not restricted to relying on machine learning for performing steps of the invention. For example, some embodiments rely on other artificial intelligence models, other computer-implemented models, statistical models, mathematical models, input from a human operator, etc.

In embodiments of the invention, said method further comprises a step of calculating a positive pathology indicator probability for each of one or more of said medical test samples at least partly based on said individualized data.

In embodiments of the invention, said step of pooling a subset of said medical test samples is at least partly based on said positive pathology indicator probability.

A positive pathology indicator probability may be understood as a probability of the target pathology indicator being present in a medical test sample. This probability may be utilized when pooling a subset of the medical test samples in the test pool. For example, for large positive pathology indicator probabilities of the medical test samples, small sample numbers may be advantageous, whereas for small positive pathology indicator probabilities larger sample numbers may be advantageous.

In embodiments of the invention, said method further comprises a step of calculating a positive pool pathology indicator probability for one or more hypothetical test pools of subsets of medical test samples at least partly based on said individualized data, wherein said step of pooling a subset of said medical test samples is at least partly based on said step of calculating said positive pool pathology indicator probability.

In embodiments of the invention, said step of pooling a subset of said medical test samples is at least partly based on selecting one of said hypothetical test pools.

A positive pool pathology indicator probability may be understood as a probability of the target pathology indicator being present in a test pool.

The positive pool pathology indicator probability PPP for a test pool of n medical test samples may for example be calculated as

$$P P P = 1 - {\prod }_{i = 1}^{n} ( 1 - P P i ) ,$$

where PPi is the positive pathology indicator probability of the individual medical test samples in the pool. The use Pi notation refers to the product of the factors to the right-hand side.

Optionally, such a calculation may also take into account potential pathology indicator loads and/or dilution of the samples due to mixing.

By establishing hypothetical test pools and calculating their positive pool pathology indicator probabilities, the utilization of test resources may be improved. It may for example be easier to predict whether a second test stage is necessary.

In embodiments of the invention, said positive pathology indicator probability is based on near contact data of said associated undiagnosed individual.

In embodiments of the invention, said positive pathology indicator probability of a first medical test sample of said medical test samples is further based on said individualized data of said associated undiagnosed individual associated with a second medical test sample of said medical test samples, wherein said first medical test sample and said second medical test sample are different medical test samples.

Probabilities of detecting a target pathology indicator in a medical test sample from an individual may depend on other individuals or near contacts of that individual. For example, if the target pathology indicator is an infectious virus, and an individual has been near a person infected with that virus, the probability of the individual being infected as well may be higher. Thus, the accuracy of the pathology indicator probability may be increased, which is advantageous.

Near contact data may comprise information relating to near contacts of an individual, and individualized data of these near contacts.

In embodiments of the invention, a positive detection of said target pathology indicator in said step of detecting said target pathology indicator is followed by a step of individually testing said medical test samples of said test pool.

If the target pathology indicator is detected in a test pool, the source of the target pathology indicator among the medical test samples of the test pool may be identified by individually testing the medical test samples of the test pool, which is advantageous. The individual testing may for example be performed using the same procedure as in the step of detecting the target pathology indicator by group testing the test pool.

In embodiments of the invention, a positive detection of said target pathology indicator in said step of detecting said target pathology indicator is followed by a step of pooling a subset of said medical test samples in said test pool into a secondary test pool followed by a step of detecting said target pathology indicator by group testing said secondary test pool.

By group testing a new subset, the number of medical test samples potentially containing the target pathology indicator may be iteratively reduced, which is advantageous.

In embodiments of the invention, said method comprises a step of depositing said medical test samples of said test pool in sample wells of a pooling plate.

In embodiments of the invention, said sample wells of said pooling plate are pre-grouped in one or more well groups, wherein each of said one or more well groups has a well number.

In embodiments of the invention, said one or more well groups are visually or spatially grouped into said one or more well groups on said pooling plate.

A pooling plate may be understood as a tray with a number of sample wells for receiving medical test samples, parts of medical test samples, extracts of medical test samples, or similar, such as a conventional PCR plate. Typical conventional PCR plates may for example have 8 rows and 12 columns of sample wells, leading to a total of 96 sample wells. A medical test sample may for example be deposited/placed/located in a sample well and be drawn up or at least partially removed again by a pipette.

In the context of a PCR plate or a pooling plate, a well may also be understood as a tube.

Having pre-grouped well groups may reduce risk of faults and improve logistics of group testing, which is advantageous.

A well number may be understood as the number of wells in a well group.

A well group may for example be defined by coloring, spatial location relative distance within a well group relative to distance to other wells, labeling, etc. This may be seen in contrast to conventional PCR plates, in which sample wells are homogenously distributed in a rectangular pattern. Such conventional PCR plates may for example be labelled with letters on one axis and numbers or another axis, similarly to a chess board. In embodiments of the invention, the well groups are established by an inhomogeneous distribution of sample wells on the pooling plate. In some embodiments, a well group is different from a single row or column of a rectangular array of sample wells.

In embodiments of the invention, said sample number is at least partly based on said well number of said one or more well groups.

Accordingly, the pooling can be adjusted to the physical testing conditions provided by pooling plates, which is advantageous.

In embodiments of the invention, said one or more well groups have different well numbers.

A first well group may for example have more than 10 sample wells, whereas a second well group has less than 10 sample wells.

In embodiments of the invention, said medical test samples of said test pool are deposited only within one of said one or more well groups.

By having medical test sample being deposited only within on well group, the test pool may be easier to manage in practice, and the risk of errors may be reduced, which is advantageous.

In some embodiments, several test pools are established, and the medical test samples of each of the test pools are distributed within individual well groups. E.g., medical test samples of a first test pool are deposited only within a first well group, and medical test samples of a second test pool are deposited only within a second well group.

In embodiments of the invention, said method comprises a step of mixing medical test samples of said test pool in a pooling well of said pooling plate.

In embodiments of the invention, said pooling plate comprises a pooling well for each of said one or more well groups.

By having a dedicated pooling well, different from the sample wells of the well group, the mixing of the medical test samples may be easier to manage in practice, and the risk of errors may be reduced, which is advantageous. The pooling well may be distinct from the sample wells of the well groups, e.g. by its location, labelling, shape, etc.

In embodiments of the invention, said step of mixing medical test samples is performed via fluid connections from said pooling well to each sample well of a well group of said one or more well groups.

In embodiments of the invention, said fluid connections are individual fluid tunnels integrated in said pooling plate.

Alternatively, the fluid connection may be fully or partially open fluid channels, in which liquid of the medical test samples can flow from on sample well to another.

In some embodiments, the fluid tunnels are sufficiently thin that liquids flow relatively slowly, although embodiments are not restricted to a particular fluid tunnel cross section. In some embodiments, the fluid tunnels are so thin that liquid, e.g. water does not flow naturally, or just extremely slowly, e.g. less than 1 meter per hour. Here, fluid may be transferred from on sample well to another via pressure, e.g. high pressure at one sample well, of low pressure (suction) at another sample well.

Having fluid connections such as fluid tunnels or fluid channels, is advantageous, since et enables transferring medical test samples between sample wells without contact to between other apparatus (e.g. a pipette) and the medical test, which is advantages. Hence, medical test samples in sample wells can for example be mixed to enable group testing.

In embodiments of the invention, tunnel lengths of each of said fluid tunnels of a well group of said one or more well groups are substantially equal.

Having substantially equal tunnel lengths may ensure that relatively even amounts of liquid from each of the sample wells of a well group are mixed in the pooling well, which is advantageous.

In embodiments of the invention, tunnel lengths of each of said fluid tunnels of a first well group of said one or more well groups are different from tunnel lengths of each of said fluid tunnels of a second well group of said one or more well groups.

Having different tunnel lengths permits a wider range of sample well configurations, which is advantageous.

In embodiments of the invention, said step of mixing medical test samples is at least partly based on suctioning medical test samples of said test pools from a well group of said one or more wells groups into said pooling well.

In embodiments of the invention, said step of mixing medical test samples is at least partly based on suctioning medical test samples of said test pools from a well group of said one or more wells groups into said pooling well via said fluid tunnels.

Such suctioning may for example be performed by establishing a substantially airtight enclosure around a pooling well, including the pooling well in the enclosure, and applying a vacuum pressure within the enclosure, for example by use of a vacuum pump. In this context, a vacuum pressure may be understood as a pressure below atmospheric pressure. In this manner, liquids from one or more sample wells may be sucked though fluid tunnels to a pooling well.

In embodiments of the invention, said step of mixing medical test samples establishes a pooled sample, wherein said step of detecting said target pathology indicator is at least partly based on said pooled sample.

In embodiments of the invention, said method further comprises a step of controlling said medical test samples by individually detecting a quality control indicator of each of said medical test samples in said test pools.

In embodiments of the invention, said quality control indicator is a conventional internal quality control indicator. Examples of conventional internal quality control indicators and conventional internal quality controls for assays for PCR are endogenous, exogenous heterologous, exogenous homologous. An indicator may be a gene, for example an endogenous control gene.

In embodiments of the invention, said step of controlling said medical test samples is at least partly based on individually testing said medical test samples located in said sample wells.

A control of the medical test samples may ensure that the medical test samples have a quality which is sufficient for detecting a target pathology indicator. For example, if the medical test sample is based on a saliva sample, the control may ensure that the medical test sample is actually, at least partly, saliva-based, and not just water. Such an individual detection/control of a quality control indicator may for example be performed using similar methods as disclosed herein for detecting a target pathology indicator, e.g. a PCR test, western blot, ELISA, etc.

The medical test samples located in the sample wells may be individually tested for a quality control indicator, in contrast to testing the medical test samples prior to placing them in the sample wells or testing an admixture in a pooling well. By having a pooling plate providing separate sample wells and pooling wells, risk of faults may be reduced and logistics of group testing and quality control may be improved, which is advantageous.

An aspect of the invention relates to a group testing system for detecting a target pathology indicator, said group testing system comprising:
medical test samples, wherein each of said medical test samples stems from an associated undiagnosed individual and has a potential pathology indicator load of said target pathology indicator at least partly based on individualized data of said undiagnosed individual, wherein said potential pathology indicator load is indicative of an actual pathology indicator load of said target pathology indicator when said target pathology indicator is present,
a test pool which is a pooled subset of said medical test samples, wherein a potential pathology indicator load variance of said test pool is lower than a potential pathology indicator load variance of said medical test samples; and
laboratory detection apparatus configured to detect said target pathology indicator in said test pool via group testing.

A group testing system according to the invention may have any of the same advantages as the method of the invention.

Laboratory detection apparatus may for example be a thermal cycler (for PCR testing), blotting equipment, ELISA equipment, etc. It may optionally involve a pooling plate as described in this disclosure, or a conventional PCR plate.

The test pool may for example be pooled by its physical location, by labelling, or digitally on a computer, e.g. as a list of medical test samples in the test pool readable on the computer.

In embodiments of the invention, said test pool is established at least partly based on said potential pathology indicator load, such that said potential pathology indicator load variance of said test pool is lower than said potential pathology indicator load variance of said medical test samples.

In embodiments of the invention, at least a part of said medical test samples of said test pool are mixed in an admixture and said laboratory detection apparatus is configured to detect said target pathology indicator in said admixture.

In embodiments of the invention, said admixture is located in said laboratory detection apparatus.

In embodiments of the invention, said laboratory detection apparatus comprises a pooling plate, wherein individual sample wells of said pooling plate comprises individual medical test samples of said test pool.

In embodiments of the invention, said laboratory detection apparatus comprises a pooling plate, wherein individual sample wells of a well group of one or more well groups of said pooling plate comprises individual medical test samples of said test pool.

In embodiments of the invention, said individual medical test samples of said test pool are mixed in a pooling well of said pooling plate.

In embodiments of the invention, said laboratory detection apparatus comprises an automatized mechanical manipulator.

In embodiments of the invention, said automatized mechanical manipulator is configured to mix said medical test samples of said test pool in said pooling well.

In embodiments of the invention, said automatized mechanical manipulator is configured to mix said medical test samples based on locations of sample wells of said one or more well groups.

The locations of sample wells of a well group may for example be programmed into the automatized mechanical manipulator, such that the automatized mechanical manipulator can mix the medical test samples of this well group, when medical test samples are placed in the sample wells.

In embodiments of the invention, said automatized mechanical manipulator is configured to mix said medical test samples of said test pool in said pooling well via suction through fluid tunnels fluidly connecting said pooling well with said sample wells of a well group of said one or more well groups.

In embodiments of the invention, said automatized mechanical manipulator is a delta robot.

An automatized mechanical manipulator may for example may for example be a robot. A robot may for example be a parallel robot/manipulator, an industrial robot arm, a collaborative robot arm, or a SCARA robot.

An automatized mechanical manipulator may have a tool/end effector configured to move, transfer, and/or mix medical test samples. Such transferring and mixing may for example be at least partly facilitated by a pooling plate.

A tool may for example be based on a pipette and/or air pressure for suction and/or pressuring liquids of medical test samples.

Using an automatized mechanical manipulator to mix medical test samples is advantageous, since the invention propose different test pools of varying sample numbers, which may be difficult for a human operator or laboratory technician to manage.

In embodiments of the invention, said group testing system comprises one or more processors configured to establish a digital representation of said test pool, at least partly based on said potential pathology indicator load, wherein said test pool is based on said digital representation of said test pool.

The one or more processors may for example be part of a computer architecture, capable of facilitating at least a part of the invention. Such a computer architecture may for example comprise one or more servers, workstation/user interface, digital storage/memory, executable programs, communication channels, etc.

In embodiments of the invention, said digital representation of said test pool is established based on a pre-established correlation between said individualized data and said potential pathology indicator load associated with said one or more processors.

In embodiments of the invention, said one or more processors are associated with a machine learning algorithm.

In embodiments of the invention, said pre-established correlation between said individualized data and said potential pathology indicator load is established by said machine learning algorithm.

In embodiments of the invention, said digital representation of said test pool is established by said machine learning algorithm.

In embodiments of the invention, said automatized mechanical manipulator is controlled based on said digital representation of said test pool to locate said medical test sample of said test pool in sample wells of said pooling plate, for example in sample wells of a well group of said one or more well groups.

In embodiments of the invention, said automatized mechanical manipulator is controlled based on said digital representation of said test pool to mix said medical test sample of said test pool into said admixture.

By communication from the one or more processors to the automatized mechanical manipulator, the arrangement of medical test samples in sample wells of a pooling plate may be communicated to the automatized mechanical manipulator. Upon receiving such communication, the automatized mechanical manipulator can be operated according to pre-programmed (fully or partly) movement patterns which engage the automatized mechanical manipulator with the correct sample wells.

In embodiments of the invention, said group testing system is configured to perform any of the method steps of the invention.

An aspect of the invention relates to a pooling plate for facilitating group testing, said pooling plate comprising:
one or more well groups of sample wells, each of said one or more well groups for test pools of medical test samples; and
one or more pooling wells, wherein each of said pooling wells is individually fluidly coupled to in individual well group of said one or more well groups via fluid connections, such as fluid tunnels.

In embodiments of the invention, said pooling plate is the pooling plate of a method of the invention.

A pooling plate according to the invention may have any of the advantages described herein, where applicable. It may for example reduce risk of faults and improve logistics of group testing.

An aspect of the invention relates to use of a pre-estimated potential pathology indicator load of a medical test sample to pool said medical test sample in a test pool for group testing, wherein said potential pathology indicator load is based on individualized data of an undiagnosed individual associated with said medical test sample.

An aspect of the invention relates to use of different test pools with different potential pathology indicator load variances to group medical test samples.

Use according to the invention may for example have any of the advantages of the method or the system of the invention.

An aspect of the invention relates to a method for treating of a pathology in a group of undiagnosed individuals, the method including the steps of:
providing medical test samples for detecting a target pathology indicator of said pathology, wherein each of said medical test samples stems from an associated undiagnosed individual;
pre-estimating a potential pathology indicator load of a target pathology indicator in each of said medical test samples at least partly based on individualized data of said associated undiagnosed individual, wherein said potential pathology indicator load is indicative of an actual pathology indicator load of said target pathology indicator when said target pathology indicator is present;
pooling a subset of said medical test samples into a test pool at least partly based on said potential pathology indicator load of each of said medical test samples to reduce a potential pathology indicator load variance of said test pool;
detecting said target pathology indicator by group testing said test pool; and
treating at least one individual of said group of undiagnosed individuals for which said target pathology indicator is detected.

In embodiments of the invention, said step of treating comprises administering an effective amount of an active pharmaceutical ingredient.

In embodiments of the invention, said active pharmaceutical ingredient is selected from active pharmaceutical ingredient(s) known to have therapeutic effect on said pathology.

In embodiments of the invention, said therapeutic effect is curative and/or palliative.

In embodiments of the invention, said step of treating comprises administering an essential life support substance.

In embodiments of the invention, said essential life support substance is selected from the group consisting of oxygen, nutrients, aqueous solutions such as saline, and any combination thereof.

The invention relates to a method of establishing a pooling approach for medical tests to reduce the cost and the time of diagnosis comprising the steps of:
a. establishing an artificially intelligent model using a dataset of already-tested patients comprising demographic information, clinical symptoms, condition severity, other laboratory tests, details of the standard test procedure, and the result of the standard test diagnosis,
b. determining a probability prediction of an undiagnosed patient to be positive based on the artificially intelligent model,
c. determining the optimal number of pool size for obtained positive pretest probability,
d. instructing a sampling apparatus to perform pooling based on the optimal number of pool size for obtained positive pretest probability.

This method and any embodiments thereof may be combined with other embodiments of the invention.

According to an embodiment of the invention, said sampling apparatus comprises plates adapted to receive biological material.

According to an embodiment of the invention, said biological material comprises be DNA, RNA, or proteins.

According to an embodiment of the invention, the method further comprises repeating the method after an approved diagnosis of at least one patient.

### THE DRAWINGS

Various embodiments of the invention will in the following be described with reference to the drawings where

FIG. 1 illustrates, schematically, a group testing system according to an embodiment of the invention,

FIG. 2 illustrates, schematically, another group testing system according to an embodiment of the invention,

FIG. 3 illustrates a conventional PCR plate according to prior art,

FIG. 4 illustrates an exemplary pooling plate according to an embodiment of the invention,

FIG. 5a-d illustrate cross-sectional views of various connections between sample wells and pooling wells of a pooling plate according to embodiments of the invention,

FIG. 6 illustrates another pooling plate according to an embodiment of the invention,

FIG. 7 illustrates method steps according to an embodiment of the invention, and

FIG. 8 illustrates an automated mechanical manipulator integrated in a group testing system according to an embodiment of the invention.

### DETAILED DESCRIPTION

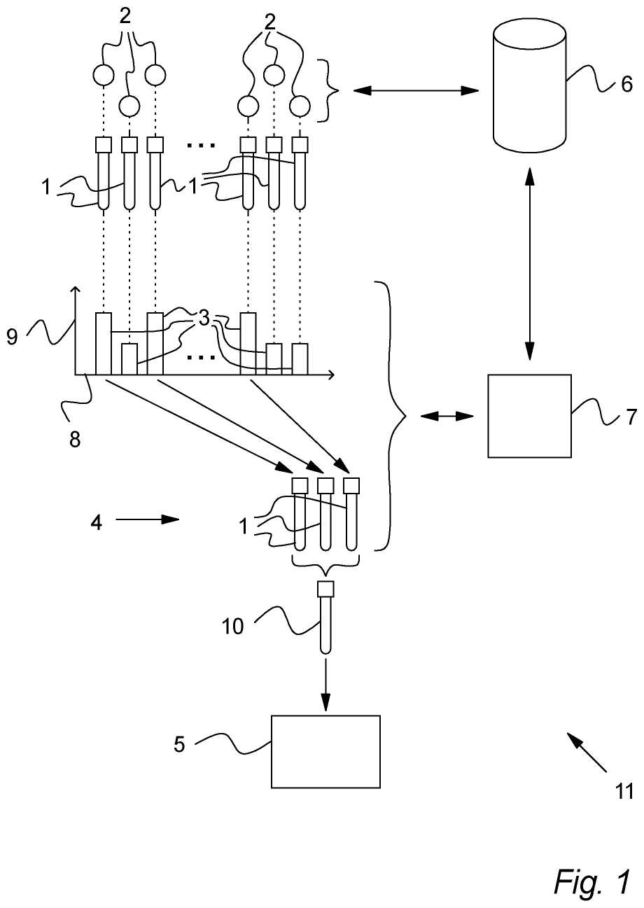

FIG. 1 illustrates, schematically, a group testing system 11 according to an embodiment of the invention. The illustrated group testing system 11 can be used to facilitate a method of the invention.

The system comprises medical test samples 1, which are to be tested to detect a target pathology indicator. In this embodiment, the target pathology indicator is SARS-CoV-2. Each of the medical test samples 1 is associated with a respective undiagnosed individual, and have been obtained from a throat swab.

The system 11 further comprises a digital storage 6, in which individualized data 2 is stored. The digital storage 6 is implemented in as part of a computer architecture used to partly facilitate this embodiment.

The individualized data 2 relates to each of the medical test samples 1 and the associated undiagnosed individual, as indicated by dotted lines. That is, a first individualized data part relates to a first medical test sample and a first undiagnosed individual associated with the first medical test sample, a second individualized data part relates to a second medical test sample and a second undiagnosed individual associated with second medical test sample etc.

In this embodiment, the individualized data 2 is indicative of whether the associated undiagnosed individual has symptoms of COVID-19 or not. Optionally, the individualized data 2 can further be indicative of age, sex, and geographical location of the associated undiagnosed individual.

The system 11 also comprises a processor 7, which is part of the computer architecture. It is communicatively connected to the digital storage 6, and is thus able to access the individualized data 2.

The processor 7 pre-estimates a potential pathology indicator load 3, i.e. a potential viral load of SARS-CoV-2, for each of the medical test samples 1. At this stage, the system 11 does not know if any of the medical test samples actually contains the target pathology indicator. But the pre-estimation provides a qualified estimate of the pathology indicator load if or when at least one medical test sample does contain the target pathology indicator. The pre-estimation is performed based on the individualized data 2. When the individualized data 2 is indicative that an undiagnosed individual has symptoms of COVID-19, the potential pathology indicator load 3 of the medical test sample 1 associated with that individual is set at a high value. And when the individualized data 2 is indicative that an undiagnosed individual does not have symptoms of COVID-19, the potential pathology indicator load 3 of the medical test sample 1 associated with that individual is set at a low value.

This pre-estimation of the potential pathology indicator loads 3 is illustrated in the figure using a bar graph having a medical test sample axis 8 and a potential pathology indicator load axis 9, indicating whether each pre-estimated potential pathology indicator load has a high value or a low value. In this exemplary illustration, the left-most medical test sample 1 has been associated with a potential pathology indicator load 3 of high value, the next medical test sample 1 has been associated with a potential pathology indicator load 3 of low value etc.

The processor 7 then selects a subset of the medical test samples 1 to be pooled in a test pool 4, based on the potential pathology indicator loads 3. In this embodiment, each medical test sample 1 with a high value of the potential pathology indicator load 3 is selected for the test pool 4, as indicated by arrows from the bar graph to the test pool 4. The selection of medical test samples 1 for the test pool 4 is performed digitally by the processor 7, but physically, the test pool is established by a human laboratory technician. This technician receives information relating to the test pool from the processor via a computer architecture interface (e.g. a computer monitor or touch screen), and this information permits the technician to hand-pick the selected medical test samples 1.

As a consequence of the pooling of a subset of the medical test samples 1 into a test pool 4, the potential pathology indicator load variance of the test pool is reduced. That is, calculating the variance VAR based on the potential pathology indicator loads 3 of medical test samples 1 in the test pool 4 is yields a smaller variance VAR than when calculating the variance VAR based on the potential pathology indicator loads 3 of all of the medical test samples 1.

A part of each of the medical test samples 1 in the test pool 4 is then mixed to establish a medical test sample admixture 10. This admixture 10 is transferred to a conventional thermal sampler, capable of performing polymerase chain reaction cycles on the admixture to potentially detect the target pathology indicator, i.e. SARS-CoV-2.

If a medical test sample 1 of the test pool 4 is positive, i.e. contains the target pathology indicator, the probability of a high value of the actual pathology indicator load is increased, since only medical test samples 1 from symptomatic individuals have been mixed in the admixture 10. Hence, by pre-estimating a potential pathology indicator load, the risk of not detecting the presence of the target pathology indicator is advantageously minimized.

The medical test samples 1 not in the test pool 4 can optionally be tested individually or in other auxiliary test pools which may for example have a smaller number of medical test samples.

The embodiment illustrated in FIG. 1 is partly computer implemented, but note that embodiments of the invention are not restricted to being computer implemented. For example, the individualized data 2 of a medical test sample 1 can be printed onto, attached, or laid into a container of that medical test sample, e.g. on a piece of paper. The medical test samples can then be assigned a potential pathology indicator and sorted by hand, for example according to pre-established logical instructions and/or calculations. Finally, the medical test samples can be pooled in test pools as large as possible, while retaining the potential pathology indicator load of each of the medical test samples above a potential load dilution threshold. Consequently, medical test samples having a large potential pathology indicator load are pooled in larger test pools than medical test samples having a small potential pathology indicator load.

Further, note that even though the above-presented exemplary embodiment was directed at detecting SARS-CoV-2, it may be used for any target pathology indicator within the scope of the claims of the invention, for example as exemplified within this disclosure.

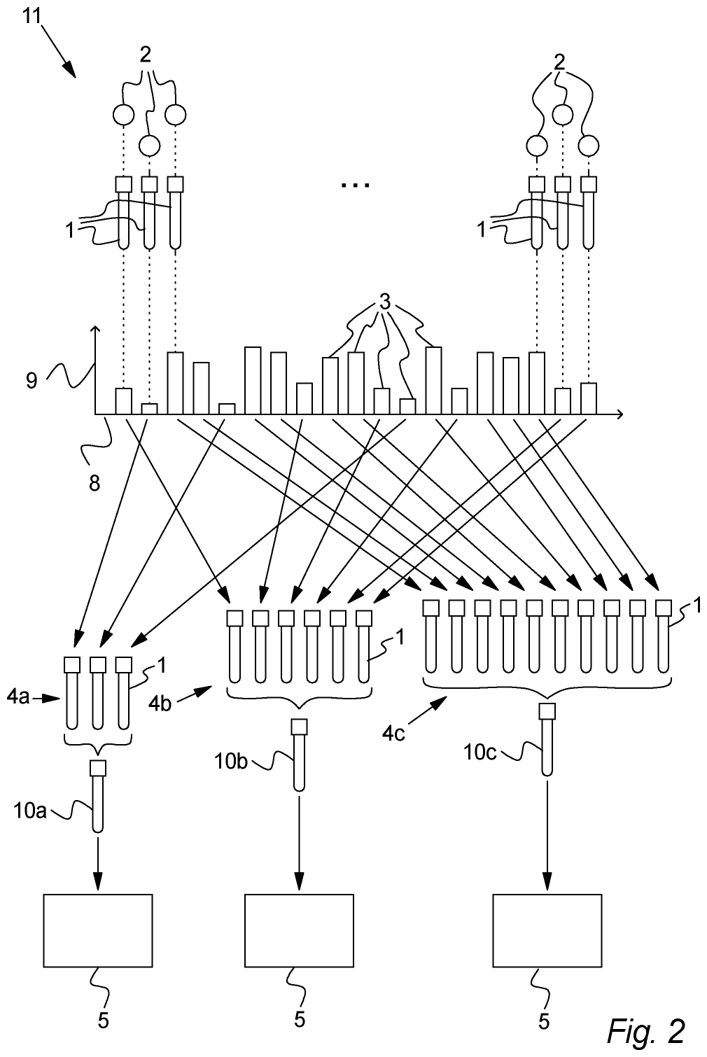

FIG. 2 illustrates, schematically, another group testing system 11 according to an embodiment of the invention. The embodiment has features substantially similar to features of the embodiment in FIG. 1. However, the embodiment of FIG. 2 has a more detailed pre-estimation of the potential pathology indicator load, and a more detailed pooling of several test pools. Note that this embodiment may typically be computer implemented, even though elements of a computer architecture are not illustrated.

In this embodiment, the potential pathology indicator load 3 of each of the medical test samples 1 is pre-estimated based on a pre-estimated correlation between the individualized data 2 and the potential pathology indicator load 3. This pre-estimated correlation has been established by correlating historical individualized data with historical actual pathology indicator loads.

The historical individualized data and the historical actual pathology indicator loads comprises data relating to individuals from which the target pathology indicator has previously been detected. Such a previous detection is not restricted to particular means of detection. As such, the historical individualized data does not necessarily stem from testing using embodiments of the invention but may for example have been obtained using conventional testing methods. Thus, the historical individualized data may for example be obtained through scientific studies relating to individuals in which the target pathology indicator has been found.

In practice, the historical individualized data comprises demographic data, age, sex, and individualized health data comprising disease symptoms, condition severity, and laboratory tests. And the historical actual pathology indicator loads comprise data indicative of detected actual pathology indicator loads upon testing each of the individuals of the historical individualized data.

The historical individualized data and the historical actual pathology indicator loads have been fed to a supervised learning machine learning algorithm is training data, which process the data to establish the pre-estimated correlation. The machine learning algorithm is capable of identifying both simple and complex correlations between the historical data sets, which are suitable for future predictions.

Based on the pre-estimated correlation between the historical individualized data and the historical actual pathology indicator loads, the potential pathology indicator load 3 of each of the medical test samples 1 is pre-estimated. The pre-estimation is performed by the machine learning algorithm based on the pre-estimated correlation. In this embodiment, the pre-estimation is performed using a decision tree algorithm trained by the machine learning. Alternatively, an artificial neural network trained by machine learning may be utilized to perform the pre-estimation. Selection of decision tree algorithm, neural network algorithm, or other AI-based algorithms for determining pre-estimation may for example depend on size and quality of training data. In some embodiments may training data may be selected to be most similar to the population to be tested, e.g. with respect to nationality/residency etc. In other embodiments, such parameters as e.g. nationality/residency may form an input parameter in the algorithm.

In an alternative embodiment, the pre-estimated correlation has been established by a machine learning algorithm, but the pre-estimation is not performed by the machine learning algorithm. Instead, the pre-estimated correlation serves as a foundation upon which human laboratory technicians performs the pre-estimation of a potential pathology indicator load.

In the embodiment of FIG. 2, the pre-estimation of the potential pathology indicator loads 3 is again illustrated using a bar graph having a medical test sample axis 8 and a potential pathology indicator load axis 9, indicating whether the value of each pre-estimated potential pathology indicator load. In this exemplary illustration, the potential pathology indicator loads 3 are distributed from having low values to high values.

As a next step, three subsets of the medical test samples 1 are pooled in separate test pools 4a,4b,4c. The pooling is based on pre-defined test resources. Concretely, a human laboratory technician has established that test resources corresponding to performing three tests on pooled test pools 4a,4b,4c are available. The medical test samples are then pooled such that the diluted potential pathology indicator is approximately similar across all the medical test samples upon mixing them. In the first test pool 4a, the potential pathology indicator of the medical test samples 1 is relatively low, and hence the sample number of this test pool 4a is kept low, to not dilute these medical test samples too much further. In the next test pool 4b, the potential pathology indicator of the medical test samples 1 is higher, and therefore, the sample number of this test pool 4b is kept higher, since these medical test samples 1 can tolerate more dilution while minimizing the risk of being below a limit of detection. Finally, In the third test pool 4c, the potential pathology indicator of the medical test samples 1 is even higher, and consequently, the sample number of this test pool 4c is largest, since these medical test samples 1 can tolerate even more dilution while minimizing the risk of being below a limit of detection.

As a consequence of the pooling, the potential pathology indicator load variance within each of the pools is reduced, in comparison with the variance calculated based on all of the medical test samples 1.

The pooling into three test pools is performed by a computer identifying the distribution in pools which ensures minimal variation of the diluted potential pathology indicator load across all three test pools. Such pooling may, optionally, be performed by an algorithm trained by machine learning as well.

The physical mixing of each of the test pools 4a,4b,4c into three separate admixtures 10a,10b,10c is performed by a human laboratory technician, which also provides each admixture 10a,10b,10c to laboratory detection apparatus, in which the target pathology indicator is detected, if it is present in any of the medical test samples 1.

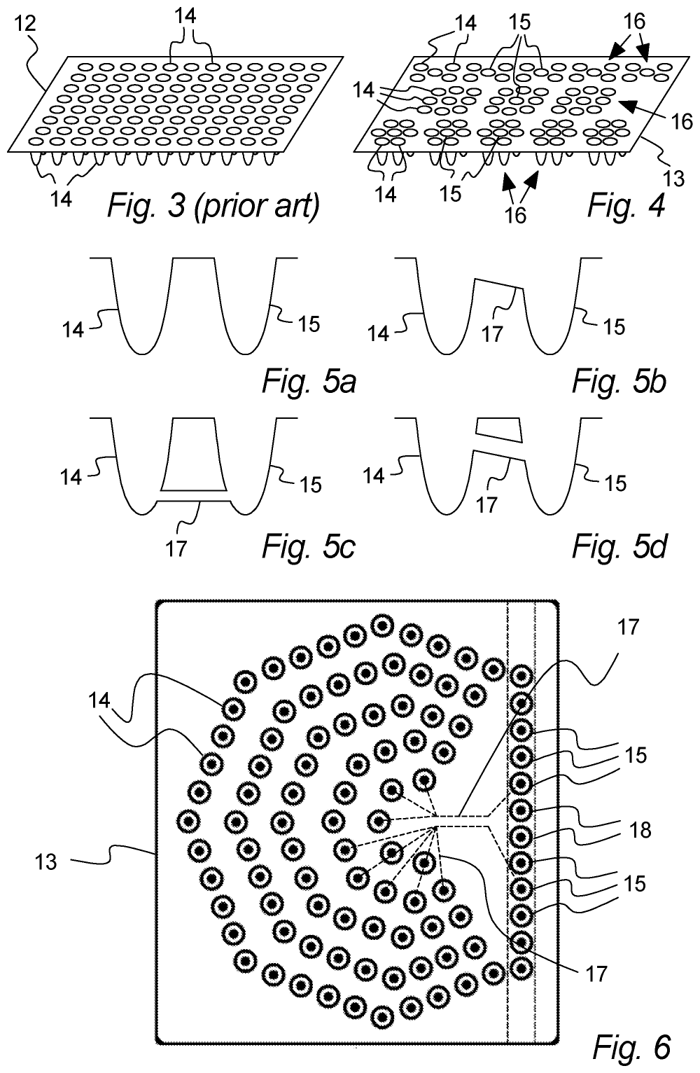

FIG. 3 illustrates a conventional PCR plate 12 according to prior art. The plate has eight rows and twelve columns of sample wells 14, resulting in a total number of 96 sample wells 14. Each of the sample wells is suitable for receiving a fluid medical test sample.

FIG. 4 illustrates an exemplary pooling plate 13 according to an embodiment of the invention.

The wells of the pooling plate 13 is grouped in well groups 16. The illustrated embodiment has five well groups 16 in a top row of groups, three well groups 16 in a middle row of groups, and five well groups 16 in a bottom row of well groups. Each well group 16 has a well number of sample wells 14 which incircles a pooling well 15 located in the center of each well group 16. Each group in the top row of well groups 16 has four sample wells 14 surrounding a central pooling well 15, each group in the middle row of well groups 16 has eight sample wells 14 surrounding a central pooling well 15, and each group in the bottom row of well groups 16 has six sample wells 14 surrounding a central pooling well 15.

A well group 16 can be used for medical test samples of a test pool, and the medical test samples can be mixed in the pooling well located with that well group.

FIG. 5a-d illustrate cross-sectional views of various connections between sample wells 14 and pooling wells 15 of a pooling plate 13 according to embodiments of the invention.

In FIG. 5a, the sample well 14 and the pooling well 15 do not have a dedicated fluid connection different from what is known from conventional PCR plates as illustrated in FIG. 3. Medical test samples may for example be transferred between wells 14,15 by a pipette or other means.

In FIG. 5b, the sample well 14 and the pooling well 15 are fluidly connected via a fluid connection 17 in the form of a fluid channel. As a medical test sample is disposed in the sample well 14, a portion of it flows to the pooling well 15 via the fluid connection 17, depending on the volume of the disposed sample. When such fluid connections 17 are used in a pooling plate, the volume of the disposed sample may be predefined to ensure that a well-defined volume of the disposed fluid flows from the sample well 14 to the pooling well 15. Similarly, the height at which the fluid connection 17 engages with each sample the wells 14,15 is selected to ensure that a particular amount of the medical test sample may be transferred. The pooling well 15 is connected to each sample well 14 of a well group and may thus receive parts of several medical test samples autonomously via fluid connections 17.

In FIG. 5c, the sample well 14 and the pooling well 15 are fluidly connected via a fluid connection 17 in the form of a fluid tunnel. The cross-sectional area of the fluid tunnel ensures that natural fluid flow from the sample well 14 to the pooling well 15 is restricted. Thus, as a fluid medical test sample is disposed in the sample well 14, it does not flow through the fluid connection autonomously. To induce flow, an airtight seal is placed on the pooling well 15 and a pump induces a vacuum in the pooling well enclosure created by the seal. Alternatively, an airtight seal is placed on the sample well 14, and a pump induces a high pressure in the sample well enclosure created by the seal. In either way, the difference in pressure between the sample well 14 and the pooling well 15 induce flow of the medical test sample from the sample well 14 to the pooling well 15. Generally, the pressure difference may be induced by any means, for example by manually squeezing a rubber bulb. By creating a vacuum at the pooling well 15, parts of medical test samples from several sample wells 14 may be transferred simultaneously to the pooling well 15.

In FIG. 5d, the sample well 14 and the pooling well 15 are fluidly connected via a fluid connection 17 in the form of a fluid tunnel. In contrast to the fluid tunnel illustrated in FIG. 5c, the fluid tunnel illustrated in FIG. 5d has a cross-sectional area which is large enough to permit natural flow. As for the embodiment in FIG. 5b, the height at which the fluid connection 17 engages with each sample the wells 14,15 is chosen to ensure that a particular amount of a medical test sample may be transferred. And again, the pooling well 15 can be connected to each sample well 14 of a well group and is thus able to receive parts of several medical test samples autonomously via fluid connections 17.

FIG. 6 illustrates another pooling plate 13 according to an embodiment of the invention.

In this embodiment, a column of pooling wells 15 and control wells 18 are located in one side of the pooling plate 13. The two middle wells in this column are control wells 18, and the rest of the wells in the column are pooling wells 15. The rest of the wells on the pooling plate 13 are sample wells 14.

Each of the pooling wells 15 are fluidly connected to a well group of sample wells 14 via fluid tunnels 17 illustrated as dashed lines. The fluid tunnels are integrated in the pooling plate, and the length of each tunnel from a certain pooling well 15 to the sample wells 14 is equal. In the figure, only connections between two pooling wells and associated samples wells are illustrated so as to not obscure the illustration with unnecessary detail. However, each of the pooling wells 15 are indeed connected to different sample wells 14 in a similar manner. Coming from the sample wells 14, the fluid tunnels 17 are grouped together midway as they approach the pooling wells 15.

The resulting pooling plate 13 has well groups of 2, 3, 5, 6, 8, 9, 11, 12, 14, and 15 sample wells, of which grouping of three sample wells and five sample wells is indicated in the illustration via the fluid connections 17. One half of the well groups is located on the top half of the plate (e.g. the well group with three sample wells 14). The other half of the well groups is located on the bottom half of the plate (e.g. the well group with five sample wells 14).

The column of pooling wells 15 and control wells 18 are located on a detachable strip of the plate 13. It is detachable via perforated lines in the plate along the column of wells.

The control wells 18 can be utilized for an overall control for the whole plate 13.

In this and other embodiments, the diameter of the tunnels are chosen as to ensure no substantial flow under atmospheric conditions. Assuming a hydrophobic surface in the wells, a temperature 20 degrees Celsius, a well depth of 2 centimeters, and a viscosity of water, a fluid will not flow under atmospheric conditions for a fluid tunnel diameter of 0.3 millimeter. By pressurizing a well, flow may then be induced. Naturally, the exact conditions for flow under atmospheric conditions may depend on well depth (defining the maximum water column), boundary layer effects (e.g. from hydrophobic or non-hydrophobic surfaces), temperature, fluid viscosity, tunnel length, tunnel shape etc. A skilled person may vary exact tunnel parameters in a multitude of ways. In some embodiments, the tunnel diameter of one or more fluid tunnels is less than 0.8 centimeter, for example less than 0.6 centimeter, for example less than 0.4 centimeter, for example less than 0.3 centimeter, for example less than 0.2 centimeter. In some alternative embodiments, one-way fluid tunnels are used, to ensure one-way flow, e.g. from sample wells to pooling wells. Such one-way fluid tunnels may for example be implemented by use of one-way valves such as duckbill valves. The height at which the fluid tunnels engage with sample wells and/or pooling wells may optionally be different for several of the individual fluid tunnels. This may ensure several fluid tunnels can overlap (seen from a top view of the plate).

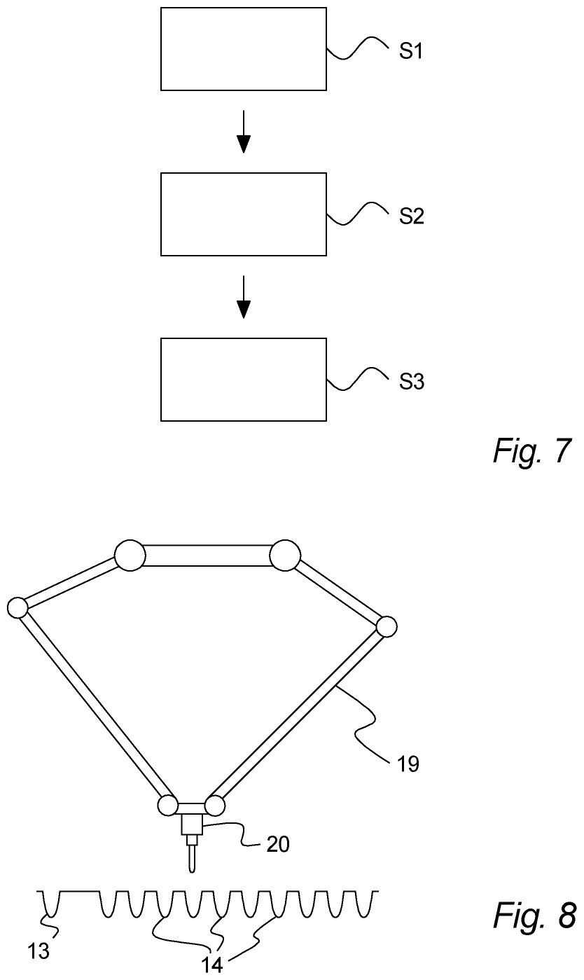

FIG. 7 illustrates method steps S1-S3 according to an embodiment of the invention.

In a first method step S1, a potential pathology indicator load of a target pathology indicator is pre-estimated for each sample of medical test samples. The pre-estimation is at least partly based on individualized data of undiagnosed individuals individually associated with each of the medical test samples. For a sample of the medical test sample, the potential pathology indicator load is indicative of an actual pathology indicator load of the target pathology indicator in case the target pathology indicator load is present in that sample.

In a next method step S2, a subset of the medical test samples a pooling into a test pool at least partly based on the potential pathology indicator load of each of the medical test samples to reduce a potential pathology indicator load variance of the test pool. The potential pathology indicator load variance is a variance of the potential pathology indicator loads of medical test samples in the test pool.

In a next step S3, the target pathology indicator is detected by group testing the test pool. This step of “detecting” may also be understood as “possibly detecting”, or “potentially detecting”, since it relies on the presence of a target pathology indicator, and prior to this step it is unknown whether the target pathology indicator is even present.

Optionally, embodiments of the invention may for example further comprise a step of pooling a subset of the medical test samples into a second test pool, a step of calculating a positive pathology indicator probability, a step of calculating a positive pool pathology indicator probability, a step of individually testing the medical test samples, a step of depositing the medical test samples, a step of mixing medical test samples, a step of controlling said medical test samples, or any combination hereof.

FIG. 8 illustrates an automated mechanical manipulator 19 integrated in a group testing system according to an embodiment of the invention.

The automated mechanical manipulator 19 is a delta robot mounted with a pipette tool 20, configured to automatically transfer medical test samples between sample wells 14 and one or more pooling wells 15.

This embodiment uses single-use pipettes which it automatically replaces between each transfer by engaging with a tray of clean pipettes.

A robot controller controlling the automated mechanical manipulator is configured to receive input as to which medical test samples are to be transferred and which wells are to be mixed. The positions of the wells have been preprogrammed into the robot controller, so upon receiving this input, it can automatically establish manipulator trajectories and tool commands for the pipette tool 20.

Optionally, the wells 14,15 or a pooling/PCR plate of the wells 14,15 are held in place by fastening means to ensure smooth operation by the robot. Fastening means may for example be clamps, a vacuum table, detachable press fit, a groove matching the plate etc.

Alternatively, the automated mechanical manipulator may have several robot tools, or other robot tools than the pipette tool.

In some embodiments of the invention, an automated mechanical manipulator has a suction cup or seal for manipulating the pressure of a well to transfer parts of medical test samples between sample wells and pooling wells via fluid connections.

In some embodiments of the invention, an automated mechanical manipulator has a gripper or jaws for decapping a tube holding or receiving a medical test sample. This may for example be performed by gripping and/or screwing.

In some embodiments, an automated mechanical manipulator has optical identification tool, such as a camera, an IR reader, or laser reader, for identifying/confirming medical test samples, or their wells/tubes.

Automated mechanical manipulators according to the invention can optionally operate based on input indicative of output of a machine learning algorithm, which determines which medical test samples should be pooled. Accordingly, the automated mechanical manipulators can mix these medical test samples, at least partly automatic manner.

### EXAMPLES

### Example 1—Study of COVID-19 Testing

In the following, a study is presented illustrating an application of the invention according to an embodiment.

The study used the large number of medical test samples of individuals being provided due to the COVID-19 pandemic. However, note that some embodiments of the invention do not necessarily include the extraction of medical test samples from individuals. Note further, that even though the testing in relation to the COVID-19 pandemic provides a platform for evaluating the invention, the invention may be used in many other pathology contexts as exemplified within this disclosure.

The study is designed as a pilot longitudinal population-based observational study to examine the feasibility of individualized group testing in the Region of Southern Denmark. During three days from 20th to 22nd of February 2021, all patients that were referring to the main testing sites in the region of the Southern Denmark were offered to fill in an anonymized questionnaire about their individual conditions. The variables under investigation were: gender, age, current condition of health, symptoms (if any) and the date of onset, history of recent contacts with a diseased individual, comorbidities, and the medication history. The inclusion criteria for the study were: i) individuals (symptomatic or asymptomatic) who are seeking a COVID-19 PCR test; ii) written consent from each individual to be included in the study. The exclusion criteria were: i) age under 18; ii) not having a CPR number (danish personal identification number); iii) an incomplete fill-out of the questionnaire.

In total, 2,360 individuals were included in the study, all of medical tests were referred to three major laboratories of: Klinisk Mikrobiologisk Afdeling (KMA), Sygehus Lillebælt, Vejle; Statens Serum Institut (SSI), Kolding; and Carelink Lab, Kolding. From the included individuals, 641 of them were excluded based on the exclusion criteria mentioned above. The remaining 1,719 individuals were all followed up to investigate on their results of COVID-19 PCR test and viral loads.

The PCR testing in all three locations were performed with nasopharyngeal swabs taken by a medical expert at the site.

From the 2,360 included patients, 39 were excluded because of a missing or wrong CPR; 356 individuals had an age below 18 years; 24 were lost to follow-up; and 222 individuals did not fill in the questionnaires completely. Hence, 641 patients were excluded from the study due to the above-outlined exclusion criteria resulting in 1,719 remaining patients in the study.

Accordingly, the nasopharyngeal swabs provided the medical test samples, and the above described variables provided individualized data associated with the medical test samples of the individuals.

The study was approved by the regional ethical committee at Region of Southern Denmark. All individuals were first orally instructed about the study, and were included in the study upon their own will after actively signing an informed consent form associated with the anonymized questionnaire. All data is anonymized and confidentiality reserved with limited access of statistical analysts.

In this specific study, Robert Dorfman's algorithm of group testing is used to determine size of groups. However, in contrast to conventional group tests, individualized groups of different sizes are established. Other algorithms may be used as well.

According to Dorfman (ref: The Detection of Defective Members of Large Populations, 1943):

$$s = \frac{{( 1 - p )}^{- s / 2}}{\sqrt{- ln ( 1 - p )}} ,$$

where p is the prevalence of the disease in the population and s is the optimal group size. Here, the positive pathology indicator probability may be used asp.

The above equation for group size s may be solved, yielding

$$s = round ( \frac{2}{Re ( W ( \frac{exp ( - {log}_{1 - p} \sqrt{- ln ( 1 - p )} ln ( 1 - p ) ) ( 1 - p )}{2} ) ) ln ( 1 - p )} ) ,$$

where W denotes the Lambert W function, Re denotes the real part of the complex number, and the “round( )” notation denotes rounding to the nearest integer.

In this example, and to reduce the potential pathology indicator load variance of the medical test samples, a machine learning algorithm is used to predict the positive pathology indicator probability of each individual, which in turn is used as the population prevalence to obtain the individualized group size for each individual based on his own positive pathology indicator probability.

The CatBoost open source software library developed by Yandex was used, which is based on gradient boosting on decision trees. Particularly, it has been developed to deal with categorical variables (hence the name CatBoost) and it does not require one-hot encoding and minimizes data preprocessing.

For training the machine learning algorithm, the training data should ideally resemble the testing data. In the case of this study, the training could not be done based on the obtained data from the Region of Southern Denmark. This was due to the very low positivity rate at the time of study, i.e. only ˜1:2000. As an alternative, the machine learning algorithm was trained with 944 heterogeneous patients from around the globe. It is to be emphasized that the results obtained herein are the most conservative showcase of the innovation, because of the limitation in obtaining more data from the Region of Southern Denmark, and training therewith.

After prediction of positive pathology indicator probabilities, the individuals were grouped. In practice, the entropy ENT was reduced using the above-mentioned variables (gender, age, current condition of health, symptoms (if any) and the date of onset, history of recent contacts with a diseased individual, comorbidities, and the medication history), where probability P relates to a positive pathology indicator probability for each patient.

According to the mathematical framework that was presented previously, it is deducible that the suggested optimal group size from a mathematical viewpoint may exceed the biological threshold of pooling samples due to dilution. There is indeed no gold standard on the pooling methodology and this comes from the fact that mathematical proposition of large group sizes ends in dilutional effect. Herein, we have rechecked the group size with the prediction of the viral loads that is based on cycle thresholds that have been obtained from the training phase.

It is to be noted that the cycle detection threshold quantities can be easily regression trained (opposed to classification) by the machine learning algorithm, and the dilutional effect can be avoided based on the cycle detection threshold predictions from the machine learning algorithm. However, in this current study, a “maximum current threshold” methodology is used to eliminate the dilutional effect. Below, a simple example is disclosed.

Let the mathematically-optimal group size s to be 32 for an individual. The extension of the machine learning algorithm then checks the training data for patients with the same condition, i.e. having a proposed group size of 32. Next, for those who have been tested positive, the cycle detection threshold values are listed in order. (That is, if one SARS-CoV-2 was detected after 34 cycles of a PCT test, the cycle detection threshold of that individual is 34 cycles.) The maximum number on the list would refer to a patient with the same clinical presentation and the least viral load. In this example, suppose the maximum cycle threshold value is less than 37. Hence, it is inferred that there is at least one patient with a similar condition who contains a viral load that may dilute if grouped in 32. The reason for this is that PCR would need 5 more cycles to create 32 (2⁵) copies from a single one. Hence, it can be inferred that after pooling, sensitivity of the test would be affected. In short, with this methodology, the machine learning algorithm always keeps a window of safety open and preserves the sensitivity based on the dynamics of viral loads.

In the following, results of the study are presented.

Regarding the narrative demographics of the individuals included in the study, the female gender dominated with 53.2% (n=916), and the mean±SD of age was 44.7±14.5. Most of the individuals (92.5%, n=1590) did not report any symptoms nor contacts. Only a small proportion of the population (5.1%, n=87) had a known contact while remaining asymptomatic. Overall, 2.4% of the individuals (n=42) were symptomatic and the most common reported symptoms were sore throat (42.9%), cough (40.5%), headache (40.5%). From the symptomatic patients, all were stable at the time of testing and no one had a severe clinical condition. 2 patients were on chronic corticosteroids and none of the patients were on any medications related to COVID-19, e.g. Remdesivir, Lopinavir/ritonavir, (Hydroxy)chloroquine, or Azithromycin.

In the follow-up of the patients and their PCR test results, only one of them turned out to be positive, who were a middle-aged woman with symptoms, yet without any known contacts. Excluding the inconclusive test results, 99.94% of the patients were negative yielding to an estimated positivity rate of approximately 1:1500 in the studied region. Such positivity rate is aligned with the findings from other studies in the literature which are performed at the same time.

To evaluate the invention, a testing scheme according to the invention was hypothetically applied to the medical test samples. The machine learning algorithm created 388 groups for the 1,719 individuals, while preserving a maximum sample number of 7 medical test samples in a given test pool. Most of the sample numbers of test pools are 3 (35.3%), 5 (23.2%), and 6 (24.5%).

In comparison with individual PCR tests, the invention permits reducing the number of PCR tests in thermal cyclers by 77.42%, thus substantially reducing the required test resources. In comparison with conventional group testing with smaller sample numbers in test polls, required test resources are also substantially reduced. In comparison with conventional group testing in groups of 3, the invention still permits a reduction of 10.7% of tests. In comparison with conventional group testing with larger sample numbers (e.g. 6), accuracy is preserved.

In some examples, the positive pathology indicator probability is pre-estimated for the target pathology indicator in each of the medical test samples, instead of the potential pathology indicator load. Then, a subset of the medical test samples is pooled into a test pool to reduce a variance of the positive pathology indicator probability in the test pool (e.g. in comparison with all medical test samples). Finally, the target pathology indicator is detected in the test pool. In other words, the positive pathology indicator may at least partly substitute the potential pathology indicator load of the invention.

From the above, it is now clear that the invention relates to a method and a system for improving group testing. By pre-estimating a potential pathology indicator in each of the medical test samples, pooling of medical test samples can be improved in a multitude of ways. Particularly, the number of medical test samples in each test pool may be maximized while minimizing the risk of declining below a limit of detection of a target pathology indicator.

The invention has been exemplified above with the purpose of illustration rather than limitation with reference to specific examples of group testing systems and methods. Details such as a specific method and system structures have been provided in order to understand embodiments of the invention for instance it is to be understood that the embodiments disclosed in the different figures and corresponding description can be combined in any way. Note that detailed descriptions of well-known systems, devices, circuits, and methods have been omitted so as to not obscure the description of the invention with unnecessary details. It should be understood that the invention is not limited to the particular examples described above and a person skilled in the art can also implement the invention in other embodiments without these specific details. As such, the invention may be designed and altered in a multitude of varieties within the scope of the invention as specified in the claims.

### LIST OF REFERENCE SIGNS

1 Medical test sample
2 Individualized data
3 Potential pathology indicator load
4 Test pool
5 Laboratory detection apparatus
6 Digital storage
7 Processor
8 Medical test sample axis
9 Potential pathology indicator load axis
10 Medical test sample admixture
11 Group testing system
12 PCR plate
13 Pooling plate
14 Sample well
15 Pooling well
16 Well group
17 Fluid connection
18 Control well
19 Automated mechanical manipulator
20 Robot tool
S1-S3 Method steps

1. A method for group testing of medical test samples for detecting a target pathology indicator, wherein each of said medical test samples stems from an associated undiagnosed individual, said method comprising the steps of:
pre-estimating a potential pathology indicator load of said target pathology indicator in each of said medical test samples at least partly based on individualized data of said associated undiagnosed individual, wherein said potential pathology indicator load is indicative of an actual pathology indicator load of said target pathology indicator when said target pathology indicator is present;
pooling a subset of said medical test samples into a test pool at least partly based on said potential pathology indicator load of each of said medical test samples to reduce a potential pathology indicator load variance of said test pool; and
detecting said target pathology indicator by group testing said test pool.

2. The method according to claim 1, wherein said test pool has a sample number of said medical test samples in said test pool.

3. The method according to claim 2, wherein said sample number is at least partly based on said potential pathology indicator load.

4. The method according to claim 2, wherein said sample number is at least partly based on a predefined amount of available test resources.

5-7. (canceled)

8. The method according to claim 1, wherein said step of pooling a subset of said medical test samples is at least partly based on retaining said potential pathology indicator load of each of said medical test samples of said test pool at least at a potential load dilution threshold.

9-15. (canceled)

16. The method according to claim 1, wherein said step of detecting said target pathology indicator by group testing said test pool is based on testing an admixture of said medical test samples in said test pool.

17. The method according to claim 1, wherein said step of detecting said target pathology indicator is based on a nucleic acid test.

18. The method according to claim 1, wherein said step of detecting said target pathology indicator is a step of repetitively performing polymerase chain reaction cycles on said test pool to detect said target pathology indicator.

19-21. (canceled)

22. The method according to claim 18, wherein said polymerase chain reaction cycles have a predefined cycle threshold at least partly based on said potential pathology indicator load of medical test samples in said test pool.

23. The method according to claim 22, wherein said cycle threshold is at least partly based on said sample number.

24-25. (canceled)

26. The method according to claim 1, wherein said target pathology indicator is a target virus, said potential pathology indicator load is a potential viral load, and said actual pathology indicator load is an actual viral load.

27. (canceled)

28. The method according to claim 1, wherein said target pathology indicator is selected from virus, bacterium, protozoan, prion, viroid, fungus, or any combination thereof.

29. The method according to claim 1, wherein said step of pooling a subset of said medical test samples comprises that said potential pathology indicator load variance is reduced in said test pool in comparison with said potential pathology indicator load variance in said medical test samples.

30-37. (canceled)

38. The method according to claim 1, wherein said individualized data comprises demographic data.

39-40. (canceled)

41. The method according to claim 1, wherein said individualized data comprises individualized health data.

42. The method according to claim 41, wherein said individualized health data comprises disease symptoms.

43-50. (canceled)

51. The method according to claim 1, wherein said pre-established correlation is at least partly based on correlating historical individualized data with historical actual pathology indicator loads.

52. (canceled)

53. The method according to claim 51, wherein said step of pre-estimating said potential pathology indicator load is performed using a machine learning algorithm trained using said historical individualized health data with historical actual pathology indicator loads.

54-81. (canceled)

82. A group testing system for detecting a target pathology indicator, said group testing system comprising:
medical test samples, wherein each of said medical test samples stems from an associated undiagnosed individual and has a potential pathology indicator load of said target pathology indicator at least partly based on individualized data of said undiagnosed individual, wherein said potential pathology indicator load is indicative of an actual pathology indicator load of said target pathology indicator when said target pathology indicator is present,
a test pool which is a pooled subset of said medical test samples, wherein a potential pathology indicator load variance of said test pool is lower than a potential pathology indicator load variance of said medical test samples; and
laboratory detection apparatus configured to detect said target pathology indicator in said test pool via group testing.

83-105. (canceled)

106. A method for treating of a pathology in a group of undiagnosed individuals, the method including the steps of:
providing medical test samples for detecting a target pathology indicator of said pathology, wherein each of said medical test samples stems from an associated undiagnosed individual;
pre-estimating a potential pathology indicator load of a target pathology indicator in each of said medical test samples at least partly based on individualized data of said associated undiagnosed individual, wherein said potential pathology indicator load is indicative of an actual pathology indicator load of said target pathology indicator when said target pathology indicator is present;
pooling a subset of said medical test samples into a test pool at least partly based on said potential pathology indicator load of each of said medical test samples to reduce a potential pathology indicator load variance of said test pool;
detecting said target pathology indicator by group testing said test pool; and
treating at least one individual of said group of undiagnosed individuals for which said target pathology indicator is detected.

107-111. (canceled)
